(19) United States
(12) Patent Application Publication
(10) Pub. No.: US 2014/0377373 A1
(43) Pub. Date: Dec. 25, 2014

(54) Compositions and Methods for the Prevention of Microbial Infections

(71) Applicants: Lankenau Institute for Medical Research, Wynnewood, PA (US); Monk Street Partners LLC, Collegeville, PA (US)

(72) Inventors: James M. Mullin, Havertown, PA (US); Jonathan Raines, Gladwyne, PA (US)

(21) Appl. No.: 14/357,641

(22) PCT Filed: Nov. 13, 2012
(86) PCT No.: PCT/US2012/064812

§ 371 Date: May 12, 2014

Related U.S. Application Data
(60) Provisional application No. 61/558,173, filed on Nov. 10, 2011.

(51) Int. Cl. A61K 33/30

(52) U.S. Cl. CPC A61K 33/30
USPC 424/642; 424/641

(57) ABSTRACT

Compositions and methods for preventing microbial infections are disclosed.

This application claims priority under 35 U.S.C. §119(e) to U.S. Provisional Patent Application No. 61/558,173, filed Nov. 10, 2011. The foregoing application is incorporated by reference herein.

### FIELD OF THE INVENTION

The present invention relates to the field of microbial infections. Specifically, compositions and methods for inhibiting and/or preventing microbial infections are disclosed.

### BACKGROUND OF THE INVENTION

Several publications and patent documents are cited throughout the specification in order to describe the state of the art to which this invention pertains. Each of these citations is incorporated herein by reference as though set forth in full.

Biomedical research over the last 10 years has revealed only a few substances, most of which are nutrients, that are capable of improving epithelial tight junction seals, and thereby decreasing leak across epithelial mucosal linings of the major organs (Amasheh et al. (2009) Ann. NY Acad. Sci., 1165:267-73). New means of regulating tight junction seals and method of inhibiting microbial pathogen entry are desired.

### SUMMARY OF THE INVENTION

In accordance with the present invention, methods of increasing tight junction barrier function and increasing transepithelial electrical resistance in an epithelial layer/sheet are provided. More particularly, the instant invention provides methods of inhibiting (reducing) and/or preventing a microbial infection in a subject. In a particular embodiment, the method comprises administering at least one composition comprising at least one zinc compound and at least pharmaceutically acceptable carrier to the epithelia of the subject. The zinc may be a pharmaceutically acceptable salt of zinc, such as zinc gluconate. In a particular embodiment, the zinc is administered topically. In a particular embodiment, the methods further comprise administering at least one other therapeutic agent or therapy for the inhibition and/or prevention of the microbial infection.

### BRIEF DESCRIPTIONS OF THE DRAWING

FIG. 1A provides a Western blot analysis showing the level of claudin-2 and the house-keeping protein β-actin (upper panels) and claudin-7 with the house-keeping protein β-tubulin (lower panels) in detergent-soluble fractions (n=3 for each condition). FIGS. 1B and 1C provide graphs of the densitometric quantification and normalization of claudin-2 and claudin-7 levels, respectively, based on house-keeping protein levels in each lane (n=3).

FIG. 2 provides graphs showing that Caco-2 cell sheets after one-week zinc supplementation have comparable short circuit currents (FIG. 2A) compared to controls (Ctrl), but exhibit a significant increase in transepithelial electrical resistance (FIG. 2B), indicating improved barrier function without alteration of active ion transport. Control cell sheets were used as 100%. (n=13-14 in each group, ***P<0.001 compared to control group or between indicated groups).

### DETAILED DESCRIPTION OF THE INVENTION

Many microbial pathogens target the tight junction (TJ) seals between epithelial cells of mucosal tissue linings. The TJ is an entry points for local and systemic infection for many microbes such as bacteria and viruses. Typically, microbial pathogens use the tight junctions as docking sites on the mucosal barrier and/or cause a loosening of the TJ barrier, thereby allowing pathogens paracellular access into the stromal region and the vasculature. Herein, it has been determined that zinc induces structural and functional changes in epithelial TJ such that the TJ barrier is improved. These structural changes render the TJ less susceptible to pathogen docking, TJ loosening, and pathogen infiltration, thereby lessening morbidity.

The linings of the skin, oral mucosa, colorectal mucosa, bladder mucosa, vaginal mucosa, and the like all constitute barriers between the external environment and the bloodstream. These linings are composed of cells connected by tight junctions (TJ). These gasket-like seals amongst the cells are, in fact, semi-permeable, thereby allowing necessary substances such as sodium, magnesium or water to permeate across. However, the tight junctions are generally not so permeable as to allow noxious substances like toxins, allergans, microbes, parasites, viruses, fungi, or bacteria from gaining entry. Disease processes ranging from inflammation to diabetes to cancer to infectious disease incorporate the weakening of these TJ seals as part of their etiology, resulting in epithelial mucosal linings that become leaky (Mullin et al. (2005) Drug Discov. Today 10:395-408). The leak in these tissue linings is not through the cells per se, but rather through the TJ seals that surround each cell of the barrier.

Microbial pathogens, e.g., viruses, bacteria, fungi, parasites (including dust mites), target the TJ apparatus during the process of infection or even as the means of infection (see, e.g., Guttman et al. (2009) Biochim. Biophys. Acta., 1788:832-41; O'Hara et al. (2008) Front Biosci., 13:7008-21). The microbial pathogens may act to disrupt and make the TJ seals leaky and/or bind to the TJ (e.g., as an entry point into the epithelial cell). Viruses which cause TJ disruption include, without limitation: HIV (Nazli et al. (2010) PLoS Pathog., 6:e1000852), echovirus (Sobo et al. (2011) J. Virol., 85:12376-86), avian influenza virus (Golebiewski et al. (2011) J. Virol., 85:10639-48), rhinovirus (Comstock et al. (2011) J. Virol., 85:6795-808; Yeo et al. (2010) Laryngoscope 120:346-52), human papilloma virus (Kranjec et al. (2011) J. Virol., 85:1757-64), SARS coronavirus (Teoh et al. (2010) Mol. Biol. Cell 21:3838-52), West Nile virus (Verma et al. (2010) Virology 397:130-8; Medigeshi et al. (2009) J. Virol., 83:6125-34), Coxsackie virus (Coyne et al. (2007) Cell Host Microbe 2:181-92; Raschperger et al. (2006) Exp. Cell Res., 312:1566-80) norovirus (Hillenbrand et al. (2010) Scand. J. Gastroenterol., 45:1307-19), herpes virus (e.g., HSV), and hepatitis C virus (HCV). It is clear that certain viruses have evolved to “open up” a mucosal barrier by making the TJ leaky, thereby allowing additional virus to enter the interstitium and systemic circulation. Indeed, many viruses have a PDZ binding domain that seeks to bind to other PDZ-domains, which are found in many TJ-associated proteins (Javier et al. (2011) J. Virol., 85:11544-56).

Notably, the TJ protein claudin-1 is required for hepatitis C virus (HCV) infection of the epithelial cell (and, thus, the organism) and the TJ protein, occludin, is a co-factor (Ahmad et al. (2011) Virol. J., 8:229; Fofana et al. (2010) Gastroenterology 139:953-64;Liu et al. (2009) J. Virol., 83:2011-4; Ciesek et al. (2011) J. Virol., 85:7613-21. The fact that the extracellular loops of claudin-1 are required for HCV entry indicates that there is interaction outside the cell between HCV and TJ proteins and that this extracellular interaction between virus and TJ is necessary for viral infection (Evans et al. (2007) Nature 446:801-5). Overall, these findings indicate that HCV binds to the TJ as part of its mechanism of entry into the epithelial cell and the organism. Accordingly, if the TJ could be structurally modified—in such a manner that is not harmful to the organism, one could make viral binding and infection less efficient or completely block viral entry, thereby reducing morbidity.

Like viruses, bacterial infections present themselves initially on the mucosal surfaces of barrier tissues (e.g., oral mucosa, nasopharyngeal mucosa, intestinal mucosa, vaginal mucosa, and the like). Certain pathogenic bacteria achieve infection in part by the disruption of TJ barriers. Example of such bacteria include, without limitation: Streptoccus pneumonia (Clarke et al. (2011) Cell Host Microbe., 9:404-14), Haemophilus influenza (Clarke et al. (2011) Cell Host Microbe., 9:404-14), Streptococcus suis (Tenenbaum et al. (2008) Brain Res., 1229:1-17), Bacillus anthracis (Bourdeau et al. (2009) J. Biol. Chem., 284:14645-56), E. coli (Denizot et al. (2012) Inflamm. Bowel Dis., 18:294-304; Strauman et al. (2010) Infect. Immun., 78:4958-64; Roxas et al. (2010) Lab Invest., 90:1152-68), Yersinia enterocolitica (Hering et al. (2011) Lab Invest., 91:310-24), Clostridium difficile (Zemljic et al. (2010) Anaerobe. 16:527-32), Neisseria miningitidis (Schubert-Unkmeir et al. (2010) PLoS Pathog. 6:e1000874, Aeromonas hydrophila (Bucker et al. (2011) J. Infect. Dis., 204:1283-92), Bacteroides fragilis (Obiso et al. (1997) Infect. Immun., 65:1431-9), and Vibrio cholera (Wu et al. (2000) Cell Microbiol., 2:11-7). All of these bacteria involve redistribution of TJ proteins and/or degradation of TJ proteins along with induction of TJ leakiness as part of their mechanism of infection. Notably, Listeria capitalizes on gaps in the epithelial barrier (at sites of cell extrusion) and then binds to basolaterally-situated E-cadherin as its docking site to the epithelial layer (Pentecost et al. (2006) PLoS Pathog., 2:e3). Accordingly, as with viruses, substances that aid in epithelial remodeling or increasing epithelial barrier integrity would inhibit bacterial colonization and/or infection.

Zinc is an active agent in certain diaper rash creams, deodorants, anti-fungal creams, calamine lotion, and anti-dandruff shampoos. Further, zinc oxide has been advocated as a therapy for topical (herpes) cold sores (Godfrey et al. (2001) Altern. Ther. Health Med., 7:49-56; Eby et al. (1985) Med. Hypotheses, 17:157-65). Specifically, the topical treatment of cold sores with a zinc oxide/glycine cream within 24 hours of onset of signs and symptoms experienced resulted in shorter duration of cold sore lesions compared to a placebo cream. It has been determined that zinc salts (e.g., zinc acetate, zinc lactate, and zinc sulfate, or zinc gluconate) directly inactivate HSV, when co-incubated.

Herein, it is demonstrated that zinc is an effective prophylactic agent in preventing disruption of epithelial linings that leads to infection by microbial agents. Used in this way, zinc not only lowers or eliminates rates of infection, but reduces the use of far more expensive remedies which become necessary once infection takes hold. Indeed, prophylactic zinc use improves health substantially by reducing leak basally and/or rendering epithelial cell layers less susceptible to microbial pathogens and/or their agents that cause TJ leak in organ linings. A prophylactically, zinc-treated epithelial tissue will be resistant to microbial infection due to the induced structural changes in the TJ. As stated hereinabove, it is demonstrated herein that zinc causes intestinal epithelia to structurally modify their TJ barriers and decrease their permeability. Zinc treatment caused statistically significant reduction of claudin-2 and, to a lesser extent, claudin-7 in intestinal tight junctions. Such modifications of the composition and structure of epithelial TJs modifies the binding of microbial pathogens to TJs and/or reduces their ability to invade epithelial cells from the region of the TJ and subsequently infect the entire organism. The zinc-induced modifications in the epithelial sheet also inhibit the formation of TJ leaks induced by pathogens. As such, the resistance of various epithelial tissues will be improved against various microbial pathogens.

The instant invention encompasses methods of inhibiting (e.g., reducing, suppressing) and/or decreasing tight junction leakage (e.g., increasing TJ barrier function and/or increasing transepithelial electrical resistance) in an epithelial layer/sheet. The methods of the instant invention comprise administering (directly or indirectly) zinc to the epithelial cells.

The instant invention also encompasses methods of inhibiting (e.g., reducing, suppressing) and/or preventing a microbial infection in a subject. Microbial infections include, without limitation, viral, bacterial, fungal, and parasitic infections. In a particular embodiment, the microbial infection is a sexually transmitted disease. The methods of the instant invention comprise administering (directly or indirectly) zinc to epithelial tissue of the subject. In a particular embodiment, the zinc is delivered or applied topically (e.g., applied to body surfaces such as the skin or mucous membranes) to the epithelial tissue. The zinc may be delivered to, for example, the skin or oral, colorectal, bladder, uterine, nasal, vaginal, penile, nasopharyngeal, buccal, or intestinal epithelial or mucosa.

In a particular embodiment, the zinc is delivered via a device (e.g., stent) or applicator to the epithelial tissue. For example, the topical compositions may be applied by an applicator such as a wipe, swab, or roller. In a particular embodiment, the zinc of the instant invention is applied to or incorporated into contraceptive devices such as a condom, diaphragm, cervical cap, intrauterine device (IUD), or vaginal sponge (e.g., contraceptive sponge) (e.g., for the inhibition of sexually transmitted microbes (e.g., HIV, etc.)). The zinc may also be administered via an implantable device such as a luminal stent, tube, or ring. The implantable medical device may be coated with a composition comprising zinc or may elute the composition. In a particular embodiment, the stent is dissolvable or degradable (e.g., a stent that exhibits substantial mass or density reduction or chemical transformation after it is introduced into a subject). In another embodiment, the stent is removable. The stent may be a sustained release device. Examples of esophageal stents include, without limitation, the Boston Scientific Ultraflex™ device, the Medtronic EsophaCoil® device, and the Cook Medical Evolution® device.

The compositions of the instant invention may be administered before, during, and/or after exposure or risk of exposure to the microbial pathogen. In a particular embodiment, the compositions of the instant invention are administered at least prior to exposure or risk of exposure to the microbial pathogen. The composition may also be administered during exposure to the microbial pathogen. In a particular embodiment, the composition is administered immediately prior to exposure to the microbial pathogen. In certain embodiments, the composition is administered within an hour or an hour, 1-3 hours, or a day prior to exposure to the microbial pathogen.

The methods may also further comprise administering at least one other therapeutic agent or therapy for the inhibition of the microbial infection. In a particular embodiment, zinc is utilized as an adjuvant/compliment to the other therapeutic agent. The other therapeutic agents or therapy may be administered consecutively and/or sequentially with the zinc therapy. In a particular embodiment, the methods further comprise the administration of at least one antimicrobial, antiviral, antifungal, antibacterial, and/or antiparasite compound. Examples of anti-fungal agents include, without limitation: terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide. Examples of anti-bacterial agents include, without limitation: antibiotics, penicillins, cephalosporins, carbacephems, cephamycins, carbapenems, monobactams, aminoglycosides, glycopeptides, quinolones, tetracyclines, macrolides, fluoroquinolones, and derivatives thereof. Examples of anti-viral agents include, without limitation: amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

As stated above, the instant invention encompasses administering zinc to a subject. The zinc may be administered as a complex with another compound. In a particular embodiment, at least one pharmaceutically acceptable salt of zinc is administered to the subject. Zinc salts include, without limitation, a zinc chelate, zinc acetate, zinc butyrate, zinc gluconate, zinc glycerate, zinc glycolate, zinc formate, zinc lactate, zinc picolinate, zinc propionate, zinc salicylate, zinc tartrate, zinc undecylenate, zinc oxide, zinc stearate, zinc citrate, zinc phosphate, zinc carbonate, zinc borate, zinc ascorbate, zinc benzoate, zinc bromide, zinc caprylate, zinc carnosine, zinc chloride, zinc fluoride, zinc fumarate, zinc gallate, zinc glutarate, zinc glycerophosphate, zinc hydroxide, zinc iodide, zinc malate, zinc maleate, zinc myristate, zinc nitrate, zinc phenol sulfonate, zinc picrate, zinc propionate, zinc selenate, zinc succinate, zinc sulfate, zinc titanate, and zinc valerate. In a particular embodiment, the zinc is administered as complexed with gluconate (zinc gluconate).

The zinc of the instant invention may be contained within a composition comprising at least one pharmaceutically acceptable carrier and, optionally, at least one additional therapeutic agent, as explained hereinabove. Alternatively, the additional therapeutic agent(s) may be contained in separate compositions comprising at least one pharmaceutically acceptable carrier. The instant invention also encompasses kits comprising at least one zinc composition as described herein and at least one composition comprising at least one additional therapeutic agent.

“Pharmaceutically acceptable” refers to entities and compositions that are physiologically tolerable and do not typically produce an allergic or similar untoward reaction when administered to an animal, particularly a human. Pharmaceutically acceptable carriers are preferably approved by a regulatory agency of the Federal or a state government or listed in the U.S. Pharmacopeia or other generally recognized pharmacopeia for use in/on animals, and more particularly in/on humans. A “carrier” refers to, for example, a diluent, adjuvant, excipient, auxiliary agent, preservative, solubilizer, emulsifier, adjuvant, stabilizing agent or vehicle with which an active agent of the present invention is administered. Common carriers include, without limitation, sterile liquids, water (e.g., deionized water), alcohol (e.g., ethanol, isopropanol), oils (including those of petroleum, animal, vegetable or synthetic origin, such as peanut oil, soybean oil, mineral oil, sesame oil and the like), Common carriers include, without limitation, water, aqueous solutions, aqueous saline solutions, aqueous dextrose solutions, aqueous glycerol solutions, oil, buffered saline, ethanol, polyol (for example, glycerol, propylene glycol, liquid polyethylene glycol and the like), dimethyl sulfoxide (DMSO), detergents, suspending agents, glucose, lactose, gum acacia, gelatin, mannitol, starch paste, magnesium trisilicate, talc, corn starch, keratin, colloidal silica, potato starch, urea, medium chain length triglycerides, dextrans, other organic compounds or copolymers and other carriers suitable for use in manufacturing preparations, in solid, semisolid, or liquid form, and suitable mixtures thereof. Suitable pharmaceutical carriers and other agents of the compositions of the instant invention are described in “Remington's Pharmaceutical Sciences” by E. W. Martin (Mack Pub. Co., Easton, Pa.) and “Remington: The Science And Practice Of Pharmacy” by Alfonso R. Gennaro (Lippincott Williams & Wilkins). The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The pharmaceutical composition of the present invention can be prepared, for example, in liquid form, or can be in dried powder form (e.g., lyophilized).

The composition may be a time release formulation. For example, the compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences (Mack Publishing Co., Easton, Pa.; Medical Applications of Controlled Release, Langer and Wise (eds.), CRC Press: Boca Raton, Fla.; Controlled Drug Bioavailability, Drug Product Design and Performance, Smolen and Ball (eds.), Wiley: New York; Ranger and Peppas (1983) J. Macromol. Sci. Rev. Macromol. Chem., 23:61; Levy et al., Science (1985) 228:190; During et al. (1989) Ann. Neurol., 25:351; Howard et al. (1989) J. Neurosurg., 71:105).

The compositions of the present invention can be administered by any suitable route. The composition may be administered systemically or directly to a desired site. In a particular embodiment, the compositions are prepared for topical administration. The composition may be administered by any suitable means including, without limitation, topical, oral, intrarectal, intranasal, and intravaginal administration. The composition for topical administration may be formulated, for example, as a suppository, enema, cream, lotion, foam, ointment, liquid, powder, salve, gel (e.g., intravaginal gel), milky lotion, drops, stick, spray (e.g., pump spray, feminine or masculine deodorant sprays), aerosol, paste, mousse, douche, or dermal patch. Types of pharmaceutically acceptable topical carriers include, without limitation, emulsions (e.g., microemulsions and nanoemulsions), gels (e.g., an aqueous, alcohol, alcohol/water, or oil (e.g., mineral oil) gel using at least one suitable gelling agent (e.g., natural gums, acrylic acid and acrylate polymers and copolymers, cellulose derivatives (e.g., hydroxymethyl cellulose and hydroxypropyl cellulose), and hydrogenated butylene/ethylene/styrene and hydrogenated ethylene/propylene/styrene copolymers), solids (e.g., a wax-based stick, soap bar composition, or powder (e.g., bases such as talc, lactose, starch, and the like), and liposomes (e.g., unilamellar, multilamellar, and paucilamellar liposomes, optionally containing phospholipids). The pharmaceutically acceptable carriers also include stabilizers, penetration enhancers (see, e.g., Remington's), chelating agents (e.g., EDTA, EDTA derivatives (e.g., disodium EDTA and dipotassium EDTA), iniferine, lactoferrin, and citric acid), and excipients. Protocols and procedures which facilitate certain formulation of the topical compositions can be found, for example, in Cosmetic Bench Reference 2005, Published by Cosmetics & Toiletries, Allured Publishing Corporation, Ill., USA, 2005 and in International cosmetic ingredient dictionary and handbook. 10th ed. Edited by Tatra E. Gottschalck and Gerald E. McEwen. Washington, Cosmetic, Toiletry and Fragrance Association, 2004.

In a particular embodiment, the composition is administered orally. The composition for oral administration may be formulated as a pill, powder, capsule, tablet (e.g., coated and uncoated, chewable), gelatin capsule (e.g., soft or hard), time-release capsule, lozenge, troche, liquid solution (e.g., gargle), buccal strips or tablets, emulsion, suspension, syrup, elixir, powders/granules (e.g., reconstitutable or dispersible), or gum. Compositions for oral administration may comprise thickeners, flavorings, diluents, emulsifiers, dispersing aids or binders.

The therapeutic agents described herein will generally be administered to a patient as a pharmaceutical preparation. The term “patient” as used herein refers to human or animal subjects. The compositions of the instant invention may be employed therapeutically, under the guidance of a physician.

The compositions comprising the zinc or other therapeutic agent of the instant invention may be conveniently formulated for administration with any pharmaceutically acceptable carrier(s). The concentration of zinc in the chosen medium may be varied and the medium may be chosen based on the desired route of administration of the pharmaceutical preparation. Except insofar as any conventional media or agent is incompatible with the zinc or other therapeutic agent to be administered, its use in the pharmaceutical preparation is contemplated.

The dose and dosage regimen of zinc or other therapeutic agent according to the invention that is suitable for administration to a particular patient may be determined by a physician considering the patient's age, sex, weight, general medical condition, and the specific condition for which the zinc or other therapeutic agent is being administered to be treated or prevented and the severity thereof. The physician may also take into account the route of administration, the pharmaceutical carrier, and the zinc or other therapeutic agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular pathological condition may be determined by dosage concentration curve calculations, as known in the art.

The pharmaceutical preparation comprising the zinc or other therapeutic agent may be administered at appropriate intervals, for example, at least twice a day or more until the pathological symptoms are reduced or alleviated, after which the dosage may be reduced to a maintenance level. The appropriate interval in a particular case would normally depend on the condition of the patient. With regard to prevention or reduction of infection, the compositions of the instant invention may be administered in doses at appropriate intervals prior to exposure to the microbial pathogen.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a prophylactically effective amount.

### Definitions

The following definitions are provided to facilitate an understanding of the present invention:

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y., 1980; and Kibbe, et al., Eds., Handbook of Pharmaceutical Excipients, American Pharmaceutical Association, Washington.

As used herein, the term “subject” refers to an animal, particularly a mammal, particularly a human.

Terms that refer to being “anti” a type of target organism (e.g., antimicrobial, antiviral, antifungal, antibacterial, antiparasite) refers to having any deleterious effects upon those organisms or their ability to cause symptoms in a host or patient. Examples include, but are not limited to, inhibiting or preventing infection, inhibiting or preventing growth or reproduction, killing of the organism or cells, and/or inhibiting any metabolic activity of the target organism. The term “antimicrobial” refers to any substance or compound that when contacted with a living cell, organism, virus, or other entity capable of replication, results in a reduction of growth, viability, or pathogenicity of that entity. As used herein the term “antibiotic” refers to a molecule that inhibits bacterial growth or pathogenesis.

As used herein, the term “prevent” refers to the prophylactic treatment of a subject who is at risk of developing a condition (e.g., microbial pathogen infection) resulting in a decrease in the probability that the subject will develop the condition.

The following examples provide illustrative methods of practicing the instant invention, and are not intended to limit the scope of the invention in any way.

### EXAMPLE

### Materials and Methods

### Analyses of Tight Junctional Proteins

Human gastrointestinal epithelial cells were allowed to grow to maximal density and re-fed with culture media at different zinc concentrations for pre-determined time points (0, 3, 6, 24 or 48 hours, or 7 days). Flasks were washed two times each with ice cold saline, then flash-frozen in an ethanol-dry ice bath, and stored at −80° C. until fractionation. At that time, flasks were quick-thawed and 2 ml of 4° C. Buffer A (20 mM Tris-HCl, pH 7.5, 0.25 M sucrose, 10 mM EGTA, 2 mM EDTA) with protease and phosphatase inhibitors (final concentrations: 0.8 μM aprotinin, 20 μM leupeptin, 50 μM bestatin, 1 mM AEBSF, 10 μM pepstatin) (Calbiochem) was added to each, cells were scraped into the buffer, the suspension mechanically disrupted and then sonicated for 60 seconds on ice and transferred to an ultracentrifuge tube. Tubes were centrifuged in a chilled Beckman 50TI rotor at 39,000 rpm for one hour at 4° C. Supernatants (“cytosolic fraction”) were discarded. To the remaining pellets, 400 μL of cold Buffer A with 1% Triton-X and protease and phosphatase inhibitors was added and pellets were mechanically broken up. Suspensions were then rocked for 90 minutes at 4° C. and centrifuged again at 39,000 rpm in a chilled Beckman 50TI rotor for one hour at 4° C. The supernatant from this final spin was the “membrane fraction.” Total protein was measured using the BioRad DC™ Protein Assay Kit.

Samples of these fractions were analyzed by polyacrylamide gel electrophoresis using a Novex XCell SureLock™ Mini-Cell apparatus and a 4-20% gradient Novex Tris-Glycine, pre-cast, 10-well, 1.5 mm thick gel (Invitrogen). Precision Plus Protein™ Kaleidoscope Standards (BioRad) were also included in each gel. Gels were run at 125 V, constant voltage, for one hour at room temperature.

Proteins were transferred from the gel to a PVDF membrane using a Novex XCell SureLock™ Mini-Cell. Transfer was run at 30 V, constant voltage, for two hours at room temperature. At the end of the transfer, to check for protein transfer efficiency, the membranes were stained with Ponceau S (Sigma) for ten minutes, destained with double-distilled water, air-dried and then photographed. The membrane was then rehydrated and washed three times for 10 minutes each with PBST (1×PBS with 0.3% Tween-20). The membranes were then blocked with 5% milk/PBST overnight at 4° C.

Blots were incubated with the specific primary antibody at a concentration of 0.3 to 1 μg/mL for one hour at room temperature. Zinc-related transport and regulatory proteins, ZnT-1 and MT-1/2, were examined with rabbit-anti-ZnT-1 (1:1000, Synaptic Systems) and mouse-anti-MT1/2 (1:50, Dako) as the primary antibodies, respectively. All tight junctional protein primary antibodies were from Zymed, Inc. The blots were then incubated with secondary antibody labeled with horseradish peroxidase along with Western Lighting chemiluminescence reagents (Perkin Elmer, Inc.). For occludin, claudin-1, -3, and -7, the secondary used was goat anti-rabbit, diluted 1:8000 in 5% milk/PBST; for claudin-2, -4, and -5 the secondary used was rabbit anti-mouse, diluted 1:6000 in 5% milk/PBST. The blots were then placed against reflection autoradiography film (Kodak) and developed in a Kodak M35A X-OMAT processor.

Films were analyzed for protein expression level by measuring optical density units with a Personal Densitometer SI (Molecular Dynamics).

Analyses of Transepithelial Electrophysiology and Permeability

Cells were seeded at the density of 5×10⁵ onto sterile Millipore Millicell polycarbonate (PCF) permeable supports (pore size 0.4 μm with a diameter of 30 mm) on Day 0. Cells were allowed to grow for 21-24 days prior to experiments (Hubatsch et al. (2007) Nat. Protoc., 2:2111-9). Three or four Millicell PCF units (2 ml/unit) were placed in a 100 mm sterile petri dish (15 ml/dish). Cells were fed bilaterally 3 times per week with control medium until at least Day 14 and switched to different zinc-supplemented media (Ctrl, 50 or 100 μM elemental zinc) for another week. In addition to this standard condition (1-week incubation with zinc), there were also two variations: 1) 2-day zinc exposure of fully differentiated cultures where cells were fed with Ctrl medium until Day 20 or 21 and switched to zinc media for another 2 days; and 2) an acute 2-hour zinc exposure on Day 21.

On the day of experiment, cells were re-fed in fresh culture medium and allowed to incubate at 37° C. for 2 hr prior to the actual experiment. Transepithelial voltage and transepithelial electrical resistance were measured as previously described (Skrovanek et al. (2007) Am. J. Physiol. Regul. Integr. Comp. Physiol., 293:R1046-55). In brief, using silver/silver chloride electrodes in series with 1M NaCl agar bridges, a 40 microamp externally applied current pulse was delivered across the cell layer and the resultant change in the voltage across the cell layer was measured using calomel electrodes in series with 1M NaCl/agar bridges and a Keithley® 197A auto-ranging digital multimeter. Ohm's law was then used to calculate transepithelial electrical resistance (Rₜ) as ohm×cm².

### Results

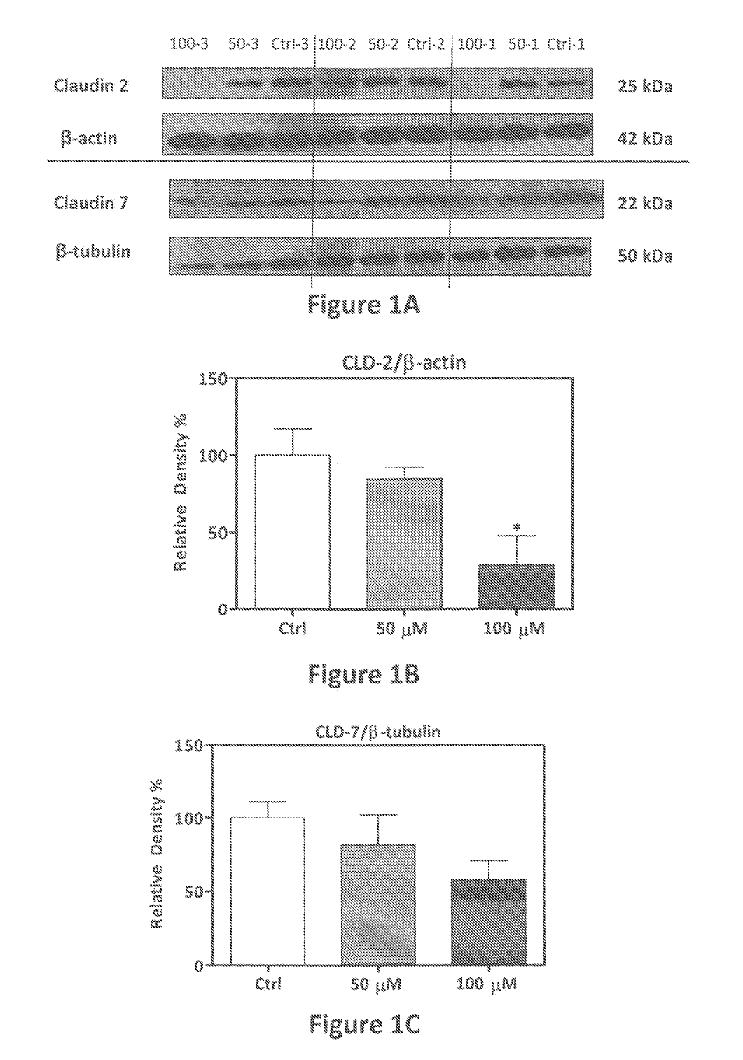

To determine the effects of zinc on the expression of tight junction (TJ) proteins, human intestinal epithelial cells (Caco-2 cell line) were incubated with zinc. Specifically, Caco-2 cells, which spontaneously form tight monolayers of polarized cells, were incubated in the presence (50 or 100 μM) or absence (control) of zinc for one week. As seen in FIG. 1, zinc altered the protein expression levels of certain TJ proteins. As seen in FIGS. 1A and 1B, zinc significantly reduced the expression of claudin-2. Further, the addition of zinc resulted in the reduction, albeit to a lesser extent than claudin-2, of the expression of claudin-7 (see FIGS. 1A and 1C).

Claudin-2 is a structural component of tight junctions in the kidneys, liver, and intestine (Sakaguchi et al. (2002) J. Biol. Chem., 277:21361-70). Claudin-2 forms a cation (Na′)-selective channel which determines the paracellular cation permeability of epithelia and Claudin-2 knockout mice are characterized by poorly developed and defective tight junctions (Amasheh et al. (2002) J. Cell Sci., 115:4969-4976; Muto et al. (2010) Proc. Natl. Acad. Sci., 107:8011-8016).

Claudin-7 promotes epithelial tightness and is found in most epithelia (Hou et al. (2006) J. Biol Chem., 281:36117-36123; Alexandre et al. (2007) Biochem. Biophys. Res. Commun., 357:87-91; Tatum et al. (2010) Am. J. Physiol. Renal Physiol., 298:F24-F34). Claudin-7 is involved in regulation of the permeability of Cl⁻ and Na⁺ ions.

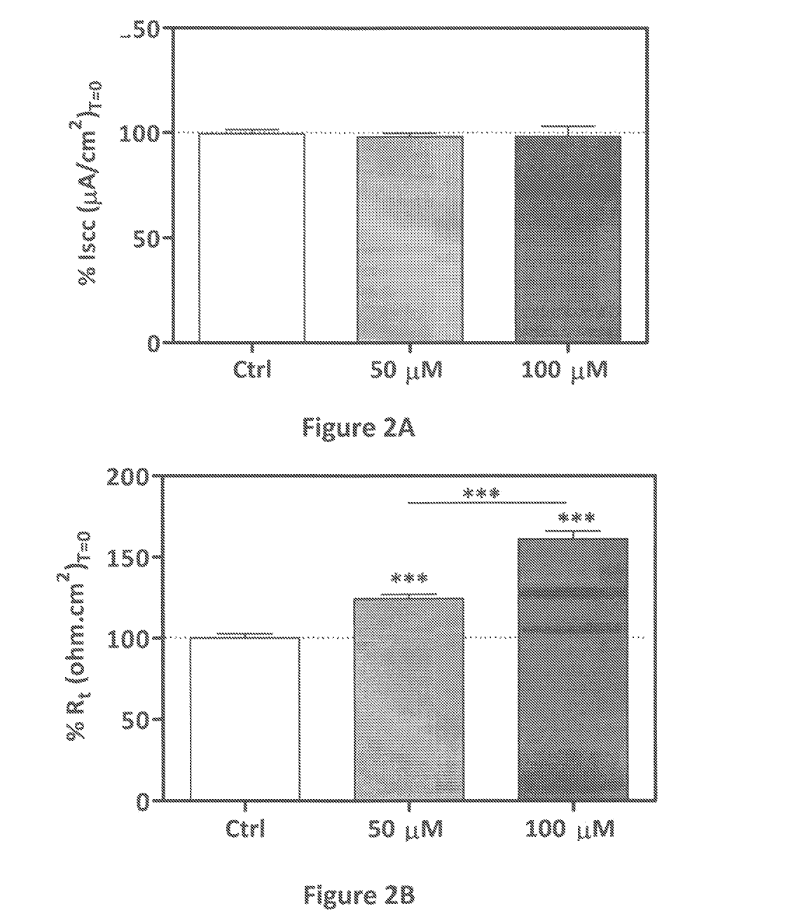

To determine the net ion transport taking place across Caco-2 cell sheets, the short-circuit current was determined after incubation in the presence (50 or 100 μM) or absence (control) of zinc for one week. As seen in FIG. 2A, comparable short circuit currents were observed regardless of the presence of zinc, indicating no alteration of active ion transport in the epithelial layer.

The tight junctions formed by these cultures were also assayed functionally by measuring transepithelial electrical resistance. After one-week zinc supplementation, the Caco-2 cell sheet exhibited a significant increase in transepithelial electrical resistance, indicating improved barrier function (FIG. 2B).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of preventing a microbial infection in a subject, said method comprising administering zinc to the epithelial tissue of said subject prior to exposure to the microbe.

2. The method of claim 1, wherein said zinc is a zinc salt.

3. The method of claim 2, wherein said zinc salt is zinc gluconate.

4. The method of claim 1, wherein said zinc is administered topically.

5. The method of claim 4, wherein said zinc is administered directly to the skin or a mucosal membrane.

6. The method of claim 4, wherein said zinc is administered to oral, colorectal, bladder, uterine, nasal, vaginal, penile, nasopharyngeal, buccal, or intestinal epithelial or mucosa.

7. The method of claim 1, further comprising administering at least one other therapeutic agent or therapy for inhibiting said microbial infection.

8. The method of claim 7, wherein said other therapeutic agent is selected from the group consisting of antivirals, antibiotics, antifungals, and antiparasitics.

9. The method of claim 1, wherein said microbe is selected from the group consisting of a virus, bacteria, fungus, and parasite.

10. The method of claim 9, wherein said virus is selected from the group consisting of HIV, echovirus, influenza virus, rhinovirus, human papilloma virus, SARS, coronavirus, coxsackie virus, norovirus, herpes, and hepatitis C virus.

11. The method of claim 9, wherein said bacteria is selected from the group consisting of Streptoccus pneumonia, Haemophilus influenza, Streptococcus suis, Bacillus anthracis, E. coli, Yersinia enterocolitica, Clostridium difficile, Neisseria miningitidis, Aeromonas hydrophila, Bacteroides fragilis, Vibrio cholera, and Listeria.

12. The method of claim 1, wherein said zinc is administered within 1-3 hours of exposure to the microbe.
